(19) United States
(12) Patent Application Publication
(10) Pub. No.: US 2004/0081682 A1
(43) Pub. Date: Apr. 29, 2004

(54) Transdermal system (tds) that contain inhibitors of phosphodiesterase lV

(76) Inventors: Clemes Guenther, Berlin (DE); Ralph Lipp, Berlin (DE); Fred Windt, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/451,225

(22) PCT Filed: Dec. 20, 2001
(86) PCT No.: PCT/DE01/04898

(30) Foreign Application Priority Data
Dec. 21, 2000 (EP) 00250450.4

(51) Int. Cl. A61K 31/42; A61K 31/4015

(52) U.S. Cl. 424/449; 424/448; 514/424; 514/376

(57) ABSTRACT

The invention relates to a transdermal system that is characterized by a content in a phosphodiesterase IV inhibitor, especially (−) rolipram or (R)-(−)-5-(4-methoxyphenyl-3-propoxy)-5-methyl-2-oxazolidinone.

This invention relates to transdermal systems that contain inhibitors of the phosphodiesterase IV, especially the more pharmacologically active (R)-(−)-enantiomer of rolipram, which is also designated as (−)-rolipram or (R)-(−)-4-(3-cyclopentyloxy-4-methylphenyl)-2-pyrrolidone), or (R)-(−)-methylphenyloxazolidinone derivatives, such as, for example, (R)-(−)-5-(4-methoxyphenyl-3-propoxy)-5-methyl-2-oxazolidinone (mesopram (INN)).

Phosphodiesterases of type IV (PDE IV) regulate the syntheses and the metabolism of cAMP. (−)-Rolipram and (R)-(−)-methylphenyloxazolidinone derivatives are inhibitors of the phosphodiesterase IV. The pharmacological activity of rolipram is extensively documented in the literature. PDE IV inhibitors can be used, i.a., for the treatment of neuropsychiatric diseases, such as, for example, depression and dementia, for influencing the secretion of gastric acid, for the relaxation of smooth muscles of the respiratory system as well as diseases induced by immunology or inflammation, especially diseases of the immune system, which are induced by stimulation of TNF and other cytokines.

Such diseases are, for example, autoimmune diseases, pulmonary diseases, infectious diseases and bone resorption diseases, such as rheumatoid arthritis, rheumatoid spondylitis, osteoarthritis, gout, sepsis, septic shock, endotoxin shock, gram-negative sepsis, toxic shock syndrome, acute respiratory distress syndrome, pulmonary high pressure and other obstructive lung diseases, cystic fibrosis, pulmonary sarcoidosis, asthma, silicosis, cachexia, colitis ulcerosa, Crohn's disease, osteoporosis, organ damage after reperfusion, inflammatory diseases of the CNS such as cerebral malaria, multiple sclerosis, panencephalitis, infectious diseases such as AIDS, bovine insanity, inflammatory diseases of the skin such as urticaria, psoriasis, atopic dermatitis, contact dermatitis, lupus erythematosus as well as diabetes insipidus as well as neuroprotection, e.g., in the case of Parkinson's disease or dementia after multiple infarctions or stroke.

This invention relates to the use of the more active (R)-(−)enantiomer of rolipram (UPAC: (R)-(−)-4-(3-cyclopentyloxy-4-methoxyphenyl)-2-pyrrolidone) of formula I

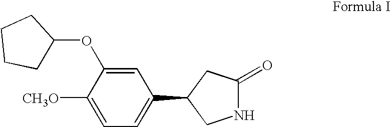

as well as (R)-(−)-methylphenyl-oxazolidinone derivatives of formula II,

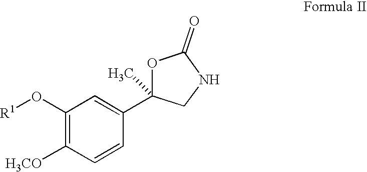

whereby R¹ means a hydrocarbon radical with 1 to 5 carbon atoms.

WO 91/09634 discloses the suitability of the racemate R/S-rolipram (CAS No. 61413-54-5) for transdermal application. Relative to this known prior art, the object of this invention is to provide crystal-free transdermal formulations of the more active (−)-enantiomer of rolipram that are easy to administer. With the technology that is described here, it has been possible, surprisingly enough, to provide an agent for transdermal application of (−)-rolipram, which compared to the use of R/S-rolipram makes possible a significantly higher crystal-free loading of the system with the more pharmacologically active enantiomer by specific use of (−)-rolipram partially in combination with suitable crystallization inhibitors. The fact that higher crystal-free loading is possible ensures larger percutaneous flows of the more active enantiomer. Thus, at the same system size, higher transdermal dosages can be administered, or a specified dose can be administered by a smaller and thus more attractive system.

WO 97/15561 discloses the suitability of methylphenyloxazolidinone derivatives for treating diseases that are mediated by TNF and by which other cytokines, for example interleukin-1 or -6, are also influenced. Production processes for enantiomer-pure methylphenyloxazolidinone derivatives are indicated, whereby especially the R derivative in comparison to the racemate is a more effective inhibitor of phosphodiesterase IV. The cerebral action in rats was observed after intraperitoneal administration, whereby the R enantiomer has proven the more effective substance.

As forms of administration, enteral or parenteral formulations are proposed that can be administered orally, sublingually or intramuscularly or intravenously or else topically or intrathecally.

Relative to this known prior art, the object of this invention is to provide crystal-free transdermal formulations that are easy to administer of those phosphodiesterase IV inhibitors, especially for (R)-(−)-5-(4-methoxyphenyl-3-propoxy)-5-methyl-2-oxazolidinone (mesopram (INN)), that allow therapeutically effective skin flows at a patch size of less than 50 cm², and with which plateau-like plasma levels can be achieved. This is important especially for (R)-(−)-5-(4-methoxyphenyl-3-propoxy)-5-methyl-2-oxazolidinone (mesopram (INN)), since this active ingredient has a narrow therapeutic range of action.

This invention achieves this object by providing transdermal systems that are suitable to pass on (−)-rolipram or (R)-(−)-5-(4-methoxyphenyl)-3-alkoxy)-5-methyl-2-oxazolidinone derivatives in the skin of a vehicle, especially a human, such that therapeutically useful skin flows result. The transdermal systems according to the invention are distinguished by a special selection of formulation components, especially adhesives, penentration intensifiers and/or crystallization inhibitors.

The transdermal system according to the invention is especially suitable for (−)-rolipram and (R)-(−)-5-(4-methoxyphenyl-3-propoxy)-5-methyl-2-oxazolidinone (mesopram (INN)).

The transdermal systems according to the invention in terms of matrix systems comprise a backing layer that is impermeable to the phosphodiesterase IV inhibitors and adjuvants and adhering thereto one to three layers of a formulation that contains the phosphodiesterase IV inhibitor in up to 30% by weight with up to 70% by weight of a medically acceptable adhesive and optionally up to 40% by weight of a penetration intensifier and optionally up to 25% by weight of crystallization inhibitor.

As a medically acceptable adhesive, for example, polyacrylate, silicone or polyisobutylene adhesives can be used. Moreover, polyurethanes, block copolymers based on styrene and other organic polymers can also be used, however.

Preferred are polyacrylate adhesives. Polyacrylate in terms of the patent is a generic term for all polymers (homo- and copolymers) that contain acrylic acid or acrylic acid derivatives. Especially preferred are vinyl acetate-acrylate copolymers and acrylate-vinyl pyrrolidone copolymers. Most preferred are heterocopolymers that consist of vinyl acetate, 2-ethylhexylacrylate and hydroxyethylacrylate (Gelva©-MPS 7881 and 7883) as well as copolymers that consist of vinylpyrrolidone and 2-ethylhexylacrylate (TSR© adhesive of the Sekisui Company).

Each of the applied layers can be coated on one or both sides with an adhesive layer, which in addition can contain penetration-intensifying and/or crystallization-inhibiting substances.

In addition, a skin contact adhesive can be attached to the side of the formulation, either covering it or around the periphery, which is not covered by the impermeable backing layer. For packing and/or storing, the accessible side of the formulation can be covered with a separating paper or a release liner.

As a backing layer, for example, 10 to 250 μm thick films that consist of polyethylene, polypropylene, polyvinyl chloride, polyvinylidene chloride and cycloolefin copolymers can be used. The latter can be metallized or painted, dyed or pigmented on one or both sides.

Release liners can be films that consist of polyethylene terephthalate, polyesters or polyethylene that can be siliconized or fluoropolymer-coated, for example, on one or both sides.

For the production and application of the formulation to the impermeable backing layer, the formulation can first work in volatile solvents, such as, for example, lower alcohols, ketones, or lower carboxylic acid esters, as well as ethanol, isopropanol, acetone or ethyl acetate, polar ethers, for example tetrahydrofuran, lower hydrocarbons, such as cyclohexane or gasoline, or else halogenated hydrocarbons, such as dichloromethane, trichloromethane, trichlorofluoroethane and trichlorofluoromethane.

As penetration intensifiers, there can be used:

Monovalent or multivalent alcohols such as ethanol, 1,2-propanediol or benzyl alcohol; saturated or unsaturated fatty alcohols with 8 to 18 carbon atoms, such as lauryl alcohol or cetyl alcohol; hydrocarbons such as mineral oil; saturated and unsaturated fatty acids with 8 to 18 carbon atoms, such as stearic acid or oleic acid; fatty acid esters with up to 24 carbon atoms or dicarboxylic acid diesters with up to 24 carbon atoms, such as methyl ester, ethyl ester, isopropyl ester, butyl ester, sec-butyl ester, isobutyl ester, tert-butyl ester or monoglyceric acid ester of acetic acid, caproic acid, lauric acid, myristic acid, stearic acid and palmitic acid, phosphatide derivatives, such as lecithin, terpenes, urea and its derivatives or ethers, such as dimethyl isosorbide and diethylene glycol monoethyl ether.

Especially preferred are lauryl alcohol, 1,2-propanediol, methyl ester and especially the isopropyl ester of myristic acid or oleic acid, diisopropyl adipate and diisopropyl sebacate, lauric acid and oleic acid, as well as mixtures thereof.

In an especially preferred embodiment, the transdermal formulation contains crystallization inhibitors that are suitable as complexing agents, for example to form solid solutions with active ingredients, to increase the interfacial solubility for the active ingredient and to reduce the tendency of the active ingredient to recrystallize after a process solvent is removed or after the temperature is reduced. The addition of crystallization inhibitors makes it possible to undertake higher active ingredient loadings of the formulation, without active ingredient crystals forming, which are available only to a very limited extent for the mass transfer into the skin.

As crystallization inhibitors, N-vinyllactam polymers, such as N-vinyl-1-aza-cycloheptan-2-one-homopolymers and N-vinyl-piperidin-2-one-homopolymers and especially polymers of vinylpyrrolidone, such as polyvidone (Kollidon®) or co-polymers of vinylpyrrolidone with vinyl acetate (copovidones), are suitable. Especially preferred is a copovidone that consists of 6 parts vinylpyrrolidone and 4 parts vinyl acetate (Kollidon® VA 64).

In terms of reservoir systems, the transdermal systems according to the invention comprise a backing layer that is impermeable to the phosphodiesterase-IV inhibitors and adjuvants and that is optionally deformed by heating and/or drawing such that it contains the phosphodiesterase IV inhibitor in up to 30% by weight with up to 70% by weight of a reservoir-forming mixture that consists of solvent or suspending agent optionally in a mixture with adjuvants, such as penetration intensifiers, crystallization inhibitors and thickening agents, whereby by bonding or gluing the above-mentioned backing layer to the reservoir it is fixed with a membrane that is permeable to the phosphodiesterase-IV inhibitor and optionally penetration intensifiers, whereby on the side of the membrane that faces away from the reservoir and faces toward the skin, a suitable medically acceptable skin contact adhesive is attached, which is provided with a removable protective layer.

As permeable membranes, for example, polymer films such as ethylene vinyl acetate copolymer or microporous polypropylene can be used.

As thickening agents, for example, substances such as hydroxypropyl cellulose, hydroxyethyl cellulose, carboxymethyl cellulose and their salts, for example, sodium salt, starches and starch derivatives, polyvinyl pyrrolidones and their derivatives as well as highly dispersed silicon dioxide and its derivatives can be used in the range of 0.1% to 50%.

As backing layers, for example, the above-mentioned can be used.

As penetration intensifiers, for example, the above-mentioned can be used, whereby they can make up to 100% of the reservoir-forming adjuvant. They are preferably admixed into the solvent or suspending agent in proportions of up to 50%.

As crystallization inhibitors, for example, the above-mentioned are used, whereby in general they can constitute up to 50% of the reservoir-forming adjuvant mixture. They are preferably added in concentrations of up to 30%.

As skin contact adhesives, for example, the above-mentioned can be used. The latter can be added to substances such as penetration intensifiers, crystallization inhibitors and tackifying additives. Tackifying additives in terms of the invention are, for example, natural, partially synthetic and synthetic resins, such as, for example, glycerol esters, such as Foral 85-E of the Hercules Company or the Unitac R 85 of the Union Camp Company, or pentaerythritol esters such as Foral 105-E, Pentalyn H-E and Permalyn 6110 of the Hercules Company, as well as Resiester N 35 of the Union Resinera Company and Westrez 2100 of the Westvaco Company, or terpene-phenolic resins, such as, for example, Dertophene T of the DRT Company.

For the production of transdermal systems of the reservoir type, the backing layer is deformed by heating or drawing, such that it is suitable for taking up a pharmaceutical substance-containing reservoir preparation. The reservoir preparation is produced by introducing the phosphodiesterase-IV inhibitor into a solvent or suspending agent that optionally contains thickening agents and/or crystallization inhibitors. It is optionally liquefied by heat, such that it can be metered volumetrically or gravimetrically in the bulge in the backing layer. Subsequently, either the permeable membrane is applied to the backing layer by bonding or gluing and then glued to a composite that consists of skin contact adhesive and release liner, or a three-layer composite that consists of permeable membrane, skin contact adhesive and release liner is applied by bonding or gluing to the backing layer. Optionally after being punched out, the individual patches that are obtained are sealed in sealed laminate bags for storage.

In this case, the transdermally effective formulation according to the invention is suitable to prepare a simple-to use formulation with a simple application, e.g., adhesion to the skin. Moreover, the formulation according to the invention is able to produce more constant plasma levels of phosphodiesterase IV inhibitors, than, for example, injected active ingredient formulations. In the especially preferred embodiment, the formulation according to the invention avoids concentration peaks of the active ingredient, which in some cases can lead to nausea in patients.

In addition, the application of the formulation according to the invention avoids first passing through the liver, by which the active ingredient concentration in the plasma can be reduced.

The invention is now explained in detail by the examples.

The production of suitable enantiomer-pure methylphenyloxazolidinone derivatives is described in WO 97/15561.

### EXAMPLE 1

### Production of a Mesopram-Transdermal System with Dimethyl Isosorbide as a Penetration Intensifier

10.0 g of (R)-(−)-5-(4-methoxyphenyl-3-propoxy)-5-methyl-2-ozaxzolidinone is dissolved with 25.5 g of dimethyl isosorbide in 50.0 g of 2-propanol in a round-bottom flask while being stirred at 55 to 60° C. Solvent that evaporates when dissolved is then supplemented. In a stirring beaker, 165.0 g of a solution of the adhesive 2-ethylhexylacrylate-N-vinyl-2-pyrrolidone-copolymer in ethyl acetate (TSR® adhesive of the Sekisui Company) is introduced, and the above-produced solution is added while being stirred. The entire batch is stirred free of air bubbles for about 30 minutes by means of a blade agitator. With knife application, the mixture that is obtained is applied to a fluoropolymer-coated polyester film (Scotchpak® 9742), so that a coating weight of 95.0 to 105.0 g of dry mass per m² is obtained. The coated films are dried at 75 to 85° C. in a drying oven to a residual solvent content of <1.2 g/m². After the drying, a polyester or polyethylene film (Cotran 9720® of the 3M Company; FORKO liners of the 4P-Film company) is laminated on. The active ingredient formulation that is now formed on both sides of the film is punched with a punching device to suitable sizes and sealed in a film bag for storage.

### EXAMPLE 2

### Production of a Mesopram-Transdermal System with Copovidone as a Crystallization Inhibitor (Adhesive: Gelva®-MPS)

In a 1 L round-bottom flask, 120.0 g of copovidone in 280.0 g of 2-propanol is dissolved under rotation at 50 to 70° C. In a 1 L stirring beaker, 37.5 g of (R)-(−)-5-(4-methoxyphenyl-3-propoxy)-5-methyl-2-oxazolidione is introduced and mixed with 375.0 g of the propanolic copovidone solution while being stirred. For homogenization, the mixture can be treated for 20 to 30 minutes in ultrasound. In a 3 L stirring beaker, 1229.5 g of an adhesive solution of a heterocopolymer mixture based on vinyl acetate and ethyl hexylacrylate (Gelva® MPS 7881) is introduced and mixed with the active ingredient-containing solution.

The batch is made up with 2-propanol to a total mass of 1800.0 g and stirred bubble-free with a blade agitator for about 30 minutes. With a continuously operating coating device, a carrier foil is coated with the above-produced mixture to a dry weight of 100+5 g/m². The coated carrier foil is dried in a two-stage drying tunnel at about 78 to 82° C. and a band rate of 15 cm per minute. Then, a separating film is laminated on, and the formulation that is coated by the films on both sides is rolled up. Round transdermal systems with a diameter of 35.6 mm are punched by means of a punching device from the rolls and sealed in air-tight bags (oxyblock).

### EXAMPLE 3

### Production of a Mesopram-Transdermal System with 1,2-Propanediol and Lauryl Alcohol as a Penetration Intensifier

In a stirring beaker, 13.5 g of 1,2-propanediol, 1.5 g of 1-lauryl alcohol and 5.0 g of (R)-(−)-5-(4-methoxyphenyl-3-propyl)-5-methyl-2-oxazolidinone are combined and dissolved in 200.0 g of 2-propanol while being stirred. 224.0 g of a solution of the adhesive 2-ethylhexylacrylate-N-vinyl-2-pyrrolidone copolymer in ethyl acetate (TSR® adhesive of the Sekisui Company)) is added to the solution and supplemented with 2-propanol to a total of 500.0 g. The solution is stirred until homogenization is complete and it is free of bubbles. Transdermal systems are produced and manufactured as described in Example 2.

### EXAMPLE 4

### Production of a Mesopram-Transdermal System with Copovidone as a Crystallization Inhibitor (TSR® adhesive of the Sekisui Company)

In a stirring beaker, 12.51 g of copovidone and 2.50 g of (R)-(−)-5-(4-methoxyphenyl-3-propoxy)-5 methyl-2-oxazolidinone are dissolved in 15.0 g of 2-propanol while being stirred. 52.45 g of a solution of the adhesive 2-ethylhexylacrylate-N-vinyl-2-pyrrolidone copolymer in ethyl acetate (TSR® adhesive of the Sekisui company) is added to this solution, and the batch is made up with 2-propanol to 90.0 g of total mass. The solution is stirred until homogenization is complete and it is free of bubbles. Then, transdermal systems are produced and manufactured as described in Example 2.

### EXAMPLE 5

### Production of a Two-Layer Mesopram-Transdermal System with Copovidone as a Crystallization Inhibitor (Adhesive: Gelva®-MPS 7881)

In a stirring beaker, 30.0 g of copovidone and 10.0 g of (R)-(−)-5-(4-methoxyphenyl-3-propoxy)-5-methyl-2-oxazolidinone are introduced and dissolved in 25.0 g of 2-propanol. 160.0 g of an adhesive solution of a heterocopolymer mixture based on vinyl acetate and ethylhexylacrylate (Gelva® MPS 7881) is added to this solution and homogenized while being stirred and stirred free of bubbles. The batch is made up to 260.0 g with 2-propanol. The mixture is applied by knife application on a separating film (Scotchpak® 9742) and dried, such that a coating produces 95.0 to 105.0 g of dry mass per m². Then, another adhesive layer is applied to the still accessible surface of the formulation without additional active ingredients or adjuvants. The layer thickness of this adhesive layer is set at 10 μm. After being dried again, a carrier foil is laminated on. Punching out and packing are performed according to Example 1.

### EXAMPLE 6

### Production of a Mesopram-Containing Reservoir Transdermal System

10.0 g of (R)-(−)-5-(4-methoxyphenyl-3-propoxy)-5-methyl-2-oxazolidinone, 20 g of polyvinylpyrrolidone (Kollidon 12 PF, of the BASF Company) and 20 g of 1,2-propanediol are dissolved in 140 g of ethanol while being heated and processed into an easily spreadable preparation with 10 g of an above-produced sodium salt of carboxymethyl cellulose (e.g., Carbopol 950 of the BF Goodrich Company). Heating and drawing deform a 200 μm thick polypropylene backing layer such that it is suitable for uptake of about 0.5 to 0.7 ml of the above-mentioned mixture on a round surface area of 10 cm². In the bulge that is obtained, 0.5 g of the above-mentioned spreadable preparation that contains 50 mg of the pharmaceutical substance is metered. Then, a three-layer laminate, produced above by coating and drying, that consists of a 50 μm thick permeable membrane that consists of ethylene vinyl acetate (Luvopor 9241, of the Lehmann Company and Voss and Co.), 50 g·m² of crosslinked polyacrylate adhesive (Gelva of the Solutia Company) and a release liner that is coated with fluoropolymer on one side (polyester film Scotchpak® 9742 of the 3M Company) are bonded, such that a circular, reservoir-free adhesive edge with a surface area of 2.5 cm² develops around the 10 cm² reservoir, and the reservoir-transdermal system thus has a total surface area of 12.5 cm². The system is punched and sealed in an oxyblock bag for storage.

### EXAMPLE 7

### Production of a (−)-Rolipram-Transdermal System with Dimethylisosorbide as a Penetration Intensifier

10.0 g of (R)-(−)-4-(3-cyclopentyloxy-4-methoxyphenyl)-2-pyrrolidone) is dissolved with 2.55 g of dimethyl isosorbide in 50.0 g of 2-propanol in a round-bottom flask while being stirred at 55 to 60° C. Solvent that evaporates when dissolved is then supplemented. In a stirring beaker, 165.0 g of a solution of the adhesive 2-ethylhexylacrylate-N-vinyl-2-pyrrolidone copolymer in ethyl acetate (TSR® adhesive of the Sekisui Company) is introduced, and the above-produced solution is added while being stirred. The entire batch is stirred free of air bubbles for about 30 minutes by means of a blade agitator. With knife application, the mixture that is obtained is applied to a fluoropolymer-coated polyester film (Scotchpak® 9742), such that a coating weight of 95.0 to 105.0 g of dry mass per m² is obtained. The coated films are dried at 75 to 85° C. in a drying oven to a residual solvent content of <1.2 g/m². After drying, a polyester or polyethylene film (Cotran 9720® of the 3M Company; FORKO liners of the 4P-Film Company) is laminated on. The active ingredient formulation that is now formed on both sides of the film is punched with a punching device to suitable sizes and sealed in film bags for storage.

### EXAMPLE 8

### Production of a (−)-Rolipram-Transdermal System with Copovidones as Crystallization Inhibitors (Adhesive: Gelva®-MPS)

In a 1 L round-bottom flask, 120.0 g of copovidone in 280.0 g of 2-propanol is dissolved at 50 to 70° C. while being rotated. In a 1 L stirring beaker, 37.5 g of (R)-(−)-4-(3-cyclopentyloxy-4-methoxyphenyl)-2-pyrrolidone) is introduced and mixed with 375.0 g of the propanolic copovidone solution while being stirred. For homogenization, the mixture can be treated for 20 to 30 minutes in ultrasound. In a 3 L stirring beaker, 1229.5 g of an adhesive solution of a heterocopolymer mixture is introduced based on vinyl acetate and ethyl hexylacrylate (Gelva® MPS 7881) and mixed with the active ingredient-containing solution.

The batch is made up with 2-propanol to a total mass of 1800.0 g and stirred bubble-free with a blade agitator for about 30 minutes. With a continuously operating coating device, a carrier foil is coated with the above-produced mixture to a dry weight of 100±5 g/m². The coated carrier foil is dried in a two-stage drying tunnel at about 78 to 82° C. and a band rate of 15 cm per minute. Then, a separating film is laminated on, and the formulation that is coated by the films on both sides is rolled up. Round transdermal systems with a diameter of 35.6 mm are punched by means of a punching device from the rolls and sealed in air-tight bags (oxyblock).

### EXAMPLE 9

### Production of a (−)-Rolipram-Transdermal System with 1,2-Propanediol and Lauryl Alcohol as Penetration Intensifiers

In a stirring beaker, 13.5 g of 1,2-propanediol, 1.5 g of 1-lauryl alcohol and 5.0 g of (R)-(−)-4-(3-cyclopentyloxy-4-methoxyphenyl)-2-pyrrolidone) are combined and dissolved in 200.0 g of 2-propanol while being stirred. 224.0 g of a solution of the adhesive 2-ethylhexylacrylate-N-vinyl-2-pyrrollidone copolymer in ethyl acetate (TSR® adhesive of the Sekisui Company)) is added to the solution and supplemented with 2-propanol to a total of 500.0 g. The solution is stirred until homogenization is complete and it is free of bubbles. Transdermal systems are produced and manufactured as described in Example 2.

### EXAMPLE 10

### Production of a (−)-Rolipram-Transdermal System with Copovidone as a Crystallization Inhibitor (TSR® Adhesive of the Sekisui Company)

In a stirring beaker, 12.51 g of copovidone and 2.50 g of (R)-(−)-4-(3-cyclopentyloxy-4-methoxyphenyl)-2-pyrrolidone) are dissolved in 15.0 g of 2-propanol while being stirred. 52.45 g of a solution of the adhesive 2-ethylhexylacrylate-N-vinyl-2-pyrrolidone copolymer in ethyl acetate (TSR® adhesive of the Sekisui Company) is added to this solution, and the batch is made up with 2-propanol to 90.0 g of the total mass. The solution is stirred until homogenization is complete, and it is free of bubbles. Then, transdermal systems are produced and manufactured as described in Example 2.

### EXAMPLE 11

### Production of a Two-Layer (−)-Rolipram-Transdermal System with Copovidone as a Crystallization Inhibitor (Adhesive: Gelva®-MPS 7881)

In a stirring beaker, 30.0 g of copovidone and 10.0 g of (R)-(−)-4-(3-cyclopentyloxy-4-methoxyphenyl)-2-pyrrolidone) are introduced and dissolved in 25.0 g of 2-propanol. 160.0 g of an adhesive solution of a heterocopolymer mixture based on vinyl acetate and ethylhexylacrylate (Gelva® MPS 7881) is added to this solution, and it is homogenized while being stirred and stirred free of bubbles. The batch is made up to 260.0 g with 2-propanol. The mixture is applied by knife application on a separating film (Scotchpak® 9742) and dried, such that a coating produces 95.0 to 105.0 g of dry mass per m². Then, another adhesive layer without additional active ingredients or adjuvants is applied to the still accessible surface of the formulation. The layer thickness of this adhesive layer is set at 10 μm. After being dried again, a carrier foil is laminated on. Punching out and packing are performed according to Example 1.

### EXAMPLE 12

### Production of a (−)-Rolipram-Containing Reservoir Transdermal System

10.0 g of (R)-(−)-4-(3-cyclopentyloxy-4-methoxyphenyl)-2-pyrrolidone), 20 g of polyvinylpyrrolidone (Kollidon 12 PF, of the BASF Company) and 20 g of 1,2-propanediol are dissolved in 140 g of ethanol while being heated and processed into an easily spreadable preparation with 10 g of an above-produced sodium salt of carboxymethyl cellulose (e.g., Carbopol 950 of the BF Goodrich Company). Heating and drawing deform a 200 μm thick polypropylene backing layer such that it is suitable for uptake of about 0.5 to 0.7 ml of the above-mentioned mixture on a round surface area of 10 cm². In the bulge that is obtained, 0.5 g of the above-mentioned spreadable preparation that contains 50 g of the pharmaceutical substance is metered. Then, a three-layer laminate, produced above by coating and drying, that consists of a 50 μm thick permeable membrane that consists of ethylene vinyl acetate (Luvopor 9241, of the Lehmann Company and Voss and Co.), 50 g·m² of crosslinked polyacrylate adhesive (Gelva of the Solutia Company) and a release liner that is coated with fluoropolymer on one side (polyester film Scotchpak® 9742 of the 3M Company) are bonded, such that a circular, reservoir-free adhesive edge with a surface area of 2.5 cm² develops around the 10 cm² reservoir, and the reservoir-transdermal system thus has a total surface area of 12.5 cm². The system is punched out and sealed in an oxyblock bag for storage.

### EXAMPLE 13

### In-Vitro Measurement of the Skin Flow of (R)-(−)-5-(4-Methoxyphenyl-3-propoxy)-5-methyl-2-oxazolidinone through the Skin of Nude Mice

After four weeks of storage at 25° C., the following transdermal systems according to the invention showed no crystal formation in the microscopic study:

TABLE 1

| TDS | Mesopram % | | Penetration | |
|---|---|---|---|---|
| Formulation | by Weight | Adhesive | Intensifier | Copovidone |
| A | 5 | 95% TSR | Without | Without |
| B | 5 | 2.5% TSR | 12.5% DMI | Without |
| C | 5 | 80% TSR | 15% PD/LA | Without |
| | | | (9 + 1) | |
| D | 10 | 75% TSR | Without | 15% |
| E | 5 | 80% Gelva | Without | 15% |

TSR* = skin contact adhesive of the Sekisui Company (ethylhexylacrylate adhesive containing 35% polymerized N-vinylpyrrolidone;
DMI = dimethyl isosorbide;
PD = propanediol:
LA = lauryl alcohol;
All formulations were produced as carrier foils on a laboratory scale with polyester film (Scotchpack ®) as a separating film and polyethylene film (Co Tran ® 9720) as a carrier foil and punched out on a surface area of 2 cm².

The skin of male nude mice (MF1 hr/hr Ola/Hsd strain of Winkelmann, Germany) at the age of 3 to 4 months was removed ventrally and dorsally to 3 cm² and after removal of attached fatty tissue, it was mounted in Franz diffusion cells. One of the formulations A to E was applied to the skin surfaces; on the tissue side, the skin of HEPES-buffered salt solution according to Hank was brought into contact with 1000 I.E. of penicillin, mixed. This acceptor solution consisted of 5.9575 g/L of HEPES, 0.35 g/L of NaHCO₃, and 0.1 L of HBSS 10× (GIBCO 032-04065, Life Technologies GmbH, Berlin) in distilled water.

Samples were taken from the acceptor liquid in the first six hours in two-hour intervals and in hours 6 to 54 in eight-hour intervals. About 1 ml of acceptor liquid per hour was pumped through the diffusion cells by means of a peristaltic pump. The entire test build-up was tempered at 31±1° C.

The amount of (R)-(−)-5-(4-methoxyphenyl-3-propoxy)-5-methyl-2-oxazolidinone that went through the skin pieces was determined by means of a radioimmunoassay.

The passage of (R)-(−)-5-(4-methoxyphenyl-3-propoxy)-5-methyl-2-oxazolidinone is depicted in Table 2, as it could be measured in the Franz diffusion cells.

TABLE 2

| Formulation | Average Flow over | Maximum Flow | tₘₐₓ |
|---|---|---|---|
| of TDS | 14 to 46 Hours (μg’ cm⁻²h⁻¹) | (μg’ cm⁻²h⁻¹) | (h) |
| A | 2.18 ± 1.03 | 2.58 ± 1.34 | 26 |
| B | 2.35 ± 0.37 | 2.51 ± 0.17 | 26 |
| C | 3.92 ± 1.73 | 4.70 ± 2.38 | 18 |
| D | 3.27 ± 2.31 | 4.16 ± 3.69 | 34 |
| E | 2.61 ± 0.72 | 3.68 ± 3.30 | 34 |

Mean value ± standard deviation: n = 4

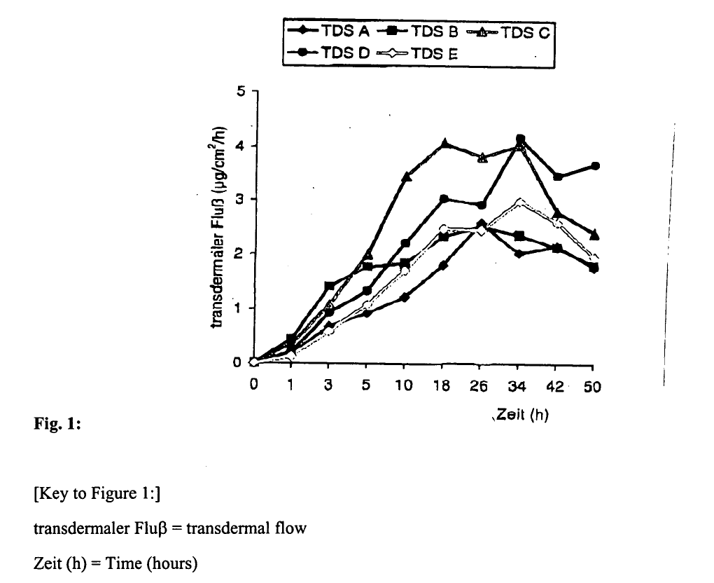

FIG. 1 shows the time plots of the mesopram flow through the mouse skin.

### EXAMPLE 14

### Pharmacokinetic Study of Humans

The above-described formulation E was tested on twelve healthy males at the age of 20 to 42 years with normal body weight, whereby for 72 hours in each case, three transdermal formulations of 10 cm² each with 5 mg of (R)-(−)-5-(4-methoxyphenyl-3-propoxy)-5-methyl-2-oxazolidinone were simultaneously applied on the lower back area. After 72 hours, the transdermal formulation was removed and within one week of washing-out time, the concentration of the active ingredient in the serum was determined per RIA. The measured serum levels of the active ingredient produced an average transdermal substance flow of 0.49±0.7 μg/cm²/h, at maximum serum levels of 0.88 ng/ml in the time interval of 29±10 hours. In particular, plateau-like plots of the serum level of the active ingredient were obtained, whereby the plateau persisted after an approximately linear increase in the first 18 hours until hour 75 and then dropped off approximately linearly.

Based on the pronounced plateau phase, which was achieved after the administration of the TDS, at the time of the reduction of TDS after three days, it was still possible to measure mesopram concentrations in the range of 65±18% of the maximum levels. After the reduction of the TDS, the serum levels with a half-life of 6.1±2.7 h dropped off. The AUC values as well as other pharmacokinetic parameters are found in Table 3. In a comparison test with intravenous administration of a total of 0.2 mg of (R)-(−)-5-(4-methoxyphenyl-3-propoxy)-5-methyl-2-oxazolidinone within one hour, a multiple of higher serum levels was measured that was 3.5 mg/ml. One hour after the infusion was completed, the mean serum levels dropped to 1.15±0.44 ng·ml·l. Later on, the serum levels dropped off four hours after completion of the infusion to achieve a level of 0.39±0.17 ng·ml·l, after 8 hours 0.17±0.09 ng·ml·l, and after 24 hours 0.08±0.08 ng·ml·l. The essential pharmacokinetic parameters after i.v. administration of mesopram are depicted in Tab. 3. The comparison test with infusion was performed on the same test subjects as the transdermal administration. In five of twelve test subjects, the infusion was brought to a halt, since they experienced nausea.

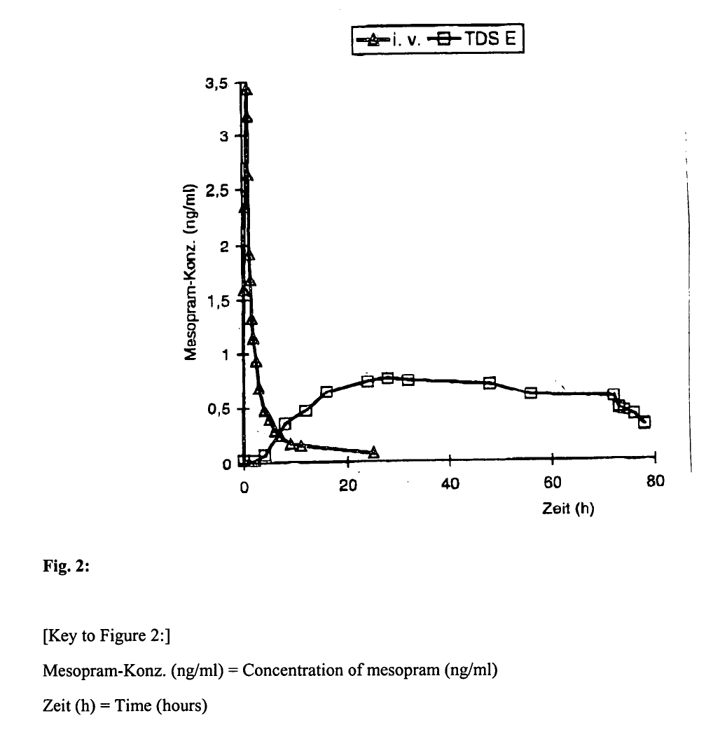

FIG. 2 shows the concentration-time plots of mesopram after transdermal administration as well as after intravenous administration.

TABLE 3
Pharmacokinetic Parameters of Mesopram-Containing
TDSE and the i.v. Reference (x ± s, n = 12)

| | Administration | |
|---|---|---|
| | Intravenous | Trandermal Administration: |
| | Infusion: 0.2 mg | 15 mg over 3 days (3 TDSE at |
| Parameters | over 1 hour | 10 cm²) |
| Cₘₐₓ (ng · ml⁻¹) | 3.6 ± 0.9 | 0.88 ± 0.22 |
| ^{t}max (h) | Without information | 29 ± 10 |
| AUC (ng · h · ml⁻¹) | 9.0 ± 3.7 | 15.8 ± 5.2* |
| ιη (h) | 2.5 ± 0.9 | 6.1 ± 2.7 |
| Cl (ml · min · kg⁻¹) | 5.1 ± 1.8 | — |
| TD (mg · d⁻¹) | — | 0.35 ± 0.12 |
| f (%) | 100 | 7 |

Cₘₐₓ—maximum concentration, tₘₐₓ—time of maximum concentration, f—absolute bioavailability, AUC=surface area under the serum curve, TD—daily dose (after transdermal administration on average), Cl=clearance, ιη=half-life of the distribution phase, o.a.—without information because of the varying lengths of the infusion periods (from 42 to 60 minutes); * per day for a total of three days; in the calculation of the transdermal doses, the individual i.v. doses were considered.

As the results depicted in FIG. 2 and Tab. 3 show, the TDSE exhibits an extraordinarily constant release of active ingredients over the period of 3 days. Since the carrying properties of the formulation on which the transdermal system E is based also allow a longer wearing time, a suitability of the obtained system is conceivable at least as twice-a-week-TDS (wearing time alternates between 3 days and 4 days). Because of the low exhaustion of the active ingredient deposit (within three days, only 7% of the active ingredient is systemically absorbed), optionally even an administration in terms of a once-a-week-TDS is conceivable.

1. Transdermal system exhibiting a content of a phosphodiesterase IV inhibitor, characterized in that the phosphodiesterase IV inhibitor is present in a matrix or in a reservoir system and in that the phosphodiesterase IV inhibitor is selected from the group below: (R)-(−)-5-(4-methoxyphenyl-3-propoxy)-5-methyl-2-oxazolidinone, in which the alkyl group contains 1 to 5 carbon atoms, or (R)-(−)-4-(3-cyclopentyloxy-4-methoxyphenyl)-2-pyrrolidone) ((−)-rolipram).

2. Transdermal system according to claim 1, wherein the phosphodiesterase IV inhibitor is (R)-(−)-5-(4-methoxyphenyl-3-alkoxy)-5-methyl-2-oxazolidinone.

3. Transdermal system according to claim 1 or 2, wherein the matrix comprises polyacrylate adhesive.

4. Transdermal system according to claim 3, wherein the polyacrylate adhesive is a copolymer of at least 2 of the following monomers: 2-ethylhexlhexylacrylate, hydroxyethylhexylacrylate, vinyl acetate, and vinyl pyrrolidone.

5. Transdermal system according to claim 4, wherein the polyacrylate adhesive is a copolymer that consists of 2-ethylhexylacrylate and hydroxyethyl-acrylate or a copolymer of these monomers with vinyl acetate and 2-ethylhexylacrylate-N-vinyl-2-pyrrolidone.

6. Transdermal system according to one of claims 1 to 5, characterized by a content of phosphodiesterase IV inhibitor of up to 30% by weight in the matrix.

7. Transdermal system according to one of claims 1 to 6, wherein the matrix consists of at least one solvent or suspending agent and the dissolved or suspended active ingredient.

8. Transdermal system according to claim 7, in which the solvent or suspending agent is ethanol or 1,2-propanediol or dimethyl isosorbide or water or mixtures of the above-mentioned substances.

9. Transdermal system according to one of claims 1 to 8, wherein the matrix or the solvent or suspending agent comprises at least one crystallization inhibitor.

10. Transdermal system according to claim 9, wherein the matrix or the solvent or the suspending agent comprises as crystallization inhibitor at least one N-vinyllactam-polymer, such as N-vinyl-1-aza-cycloheptan-2-one homopolymer, N-vinyl-piperidin-2-one homopolymer, polymers of vinyl pyrrolidone such as polyvidone (Kollidon®) or copolymers of vinyl pyrrolidone with vinyl acetate (copovidone) or highly dispersed silicon dioxide (Aevosil).

11. Transdermal system according to one of claims 1 to 10, characterized by an additional content of at least one of the following penetration intensifiers: Monovalent or multivalent alcohols such as ethanol, 1,2-propanediol or benzyl alcohol; saturated or unsaturated fatty alcohols with 8 to 18 carbon atoms, such as lauryl alcohol or cetyl alcohol; hydrocarbons such as mineral oil; saturated and unsaturated fatty acids with 8 to 18 carbon atoms, such as stearic acid or oleic acid; fatty acid esters with up to 24 carbon atoms or dicarboxylic acid diesters with up to 24 carbon atoms, such as methyl ester, ethyl ester, isopropyl ester, butyl ester, sec-butyl ester, isobutyl ester, tert-butyl ester or monoglyceric acid ester of acetic acid, caproic acid, lauric acid, myristic acid, stearic acid and palmitic acid, phosphatide derivatives, such as lecithin, terpenes, urea and its derivatives or ethers, such as dimethyl isosorbide and diethylene glycol monoethyl ether.

12. Transdermal system according to claim 11, characterized by a content of at least one of the following penetration intensifiers: lauryl alcohol, 1,2-propanediol, methyl ester and especially the isopropyl ester of myristic acid or oleic acid, diisopropyl adipate and diisopropyl sebacate, lauric acid and oleic acid, as well as mixtures thereof.
